(19) United States
(12) Patent Application Publication
(10) Pub. No.: US 2017/0100239 A1
(43) Pub. Date: Apr. 13, 2017

(54) METHOD FOR THICKNESS CONTROL AND THREE-DIMENSIONAL SHAPING OF BIOLOGICAL TISSUE DURING FIXING

(71) Applicant: BIOTRONIK AG, 8180 Buelach (CH)

(72) Inventors: Alexander Rzany, Nuernberg (DE); Wilhelm Erdbruegger, Konstanz (DE); Bernhard Hensel, Erlangen (DE)

(21) Appl. No.: 15/277,601

(22) Filed: Sep. 27, 2016

(30) Foreign Application Priority Data
Oct. 12, 2015 (DE) 102015117318.2

(51) Int. Cl. A61F 2/24

(52) U.S. Cl. CPC A61F 2/2415; A61F 2/2412; A61F 2/2418

(57) ABSTRACT

A method for impressing a 3D shape onto a biological tissue, in particular pericardial tissue, during the cross-linking of the tissue, by use of a mold which has a first 3D contact face for laminar contact against an upper side of the tissue and a second 3D contact face for laminar contact against an rear side of the tissue, wherein the tissue is arranged between the two contact faces so that it lies on both sides thereagainst and at the same time is cross-linked by means of a cross-linking agent so that the cross-linked tissue has a 3D shape after removal from the mold. The invention also relates to an implant comprising such a tissue.

### CROSS REFERENCE TO RELATED APPLICATIONS

This application claims benefit of priority to German patent application no. DE 10 2015 117 318.2 filed Oct. 12, 2015; the content of which is herein incorporated by reference in its entirety.

### TECHNICAL FIELD

The invention relates to a method for impressing a 3D shape onto a biological tissue, in particular pericardial tissue, and to an implant, in particular in the form of a heart valve prosthesis such as a heart valve.

### BACKGROUND

Solutions for the thickness control of tissue during fixing which are aimed at a fixing of the tissue between porous ceramic supports generally are already known from the prior art, such as provided in US 2013/0310929A1.

In the case of implantation of TAVI heart valves (TAVI stands for transcatheter aortic valve implantation), the diameter of the catheter system which carries the heart valve is one of the key properties in order to avoid cardiovascular complications or in order to carry out an intervention of this type in the first place. The diameter of the catheter capsule is the limiting factor here and is essentially dependent on the size of the valve stent and the thickness of the tissue used. A thickness reduction of the tissue therefore leads to a smaller spatial requirement and therefore to smaller capsule diameters, in particular since the tissue lies in folds in the crimped valve.

Current designs of heart valves that can be implanted in a minimally invasive manner consist of a metallic valve stent and a plurality of parts of biological valve tissue which form the valve cusp (leaflet) and the seal (skirt). In order to connect the biological tissue to the valve stent in a mechanically stable manner, the tissue pieces are individually sewn by hand to the valve stent and to one another. The sewing process is not performed continuously in seams with a thread, but with individual knots. Depending on the valve design, several hundreds of individual knots are necessary for a stable connection of the individual components. These knots are made by means of complex manual work using a needle, thread and scissors under microscopic observation. On account of the complex sewing process, a high level of rejection at a relatively late part of the process chain with high-quality starting components and high staff costs is practically unavoidable. Furthermore, this type of production has clear disadvantages with regard to the high cost associated with long staff working times and difficult scalability of the processes with regard to item quantities. In addition, the seam is a potential weak point with regard to the changing mechanical load over the service life of the valve implant.

### SUMMARY

On this basis, the object of the present invention is to create a method for treating biological tissue and also an implant using the tissue by which the aforementioned disadvantages are at least partly mitigated.

This object is achieved, at least in part, by a method for impressing a 3D shape onto a biological tissue, in particular pericardial tissue and preferably collagen-rich pericardial tissue, which includes cross-linking tissue arranged against two contact faces of a mold, where a first 3D contact face is for laminar contact against an upper side of the tissue, and a second 3D contact face is for laminar contact against a rear side of the tissue, wherein the tissue is arranged or further clamped or pressed between the two contact faces in such a manner that it lies tightly on both sides against these contact faces and at the same time is cross-linked by means of a cross-linking agent so that the cross-linked tissue has a 3D shape after removal from the mold.

At least one 3D contact face is a non-planar contact face, which thus has a curvature in three-dimensional space. In particular, the second 3D contact face can follow a curvature of the first 3D contact face so that a tissue with 3D shape of constant thickness results. However, the two 3D contact faces can also have a locally or generally different curvature or course so that the thickness of the cross-linked issue can be locally adjusted or controlled.

The invention thus advantageously provides three-dimensional shaping and thickness reduction or control of biological tissues, in particular for implants, heart valve prostheses, preferably TAVI heart valve prostheses, achieving objectives of a smaller catheter diameter, the possibility of new valve designs, and the reduction of required seams between biological tissue and stent for use in heart valves and other implants. In particular, heart valves made of tissue can be produced in one piece, i.e. integrally, with the method according to the invention.

In accordance with a preferred embodiment of the method according to the invention, the thickness of the cross-linked tissue is controlled or limited to a maximum value by means of a spacing between the two contact faces. The term “limited to a maximum value” means that the spacing between the contact faces constitutes the greatest possible extent of the thickness of the cross-linked tissue. In addition to the shaping, this also makes it possible to control the thickness of the tissue.

In accordance with a preferred embodiment of the method according to the invention, the mold has an upper, preferably rigid mold region, which for example has a porous material forming the first 3D contact face, and a lower mold region, which for example has a porous material forming the second 3D contact face. In a preferred embodiment the mold regions are treated with aqueous NaCl (preferably 0.9% w/v) prior to cross-linking, preferably by immersing the mold region in a solution of this type. The mold regions are preferably saturated with NaCl (preferably 0.9% w/v).

The mold regions are thus preferably permeable for the cross-linking agent so that it can pass via the mold regions to the tissue during the cross-linking and can contact the tissue.

The preferably porous mold regions or supports can have a desired or arbitrary three-dimensional shape (3D contact faces), as can be produced for example by 3D printers. The mold regions can be formed for example by open-pore polymer supports, which themselves do not react with the cross-linking agents (for example porous polycarbonate). Other suitable (porous) mold regions or support materials include, for example, sintered borosilicate glass, ceramics, or porous metals. The pore size, pore distribution, and mechanical properties of the support determine the feed of cross-linking agent.

The tissue to be cross-linked is preferably placed in a native state between the porous or permeable mold regions or supports and is fixed lying tightly thereagainst, for example with the aid of fixing (in particular by clamps or screws). The fiber proteins in the tissue are thus stabilized in the physical arrangement, space-occupying structural changes are prevented, and the three-dimensional shape is impressed.

When the tissue is cross-linked, the collagen fibers in the pericardial tissue are cross-linked by means of a suitable cross-linking agent by the incorporation of chemical bonds. The cross-linking agent binds to free amino groups of the collagen fibers and forms chemically stable connections between collagen fibers. A biological material that is stable in the long-term is thus created from the three-dimensionally arranged collagen fibers and in addition is no longer identified as foreign biological material. Due to the three-dimensional cross-linking or linking of the individual collagen fibers via the cross-linking agent, the stability and load-bearing capability of the tissue are considerably increased. This is key in particular in the case of use as tissue of a heart valve, where the tissue should open and close as a valve at approximately one-second intervals.

Due to the additional three-dimensional shaping of the tissue during the cross-linking (for example with glutaraldehyde), it is possible to produce the tissue or the biological part of the valve implant from a smaller number of tissue pieces by sewing them onto the valve stent. This is advantageous in particular since the sewing of shaped pieces is very time-consuming and therefore costly. By means of the proposed method is possible to produce valve implants in a time-saving and economical manner.

All types of tissue of mammals, including humans, can be used in principle for the proposed method. Non-human tissue is preferred. Tissues that can be used as valve material in a heart valve are particularly suitable. Pericardial tissue, in particular collagen-rich pericardial tissue, and heart valves, but also skin, ligament tissue, tendons, peritoneal tissue, dura mater, tela submucosa, in particular of the gastrointestinal tract, or lung tissue are preferred. In the case of heart valves, any valve can be used, i.e. aortic, pulmonary, mitral and tricuspid valves. Furthermore, pericardial tissues from pig, sheep, goat, horse, crocodile, kangaroo, ostrich and cattle are preferred.

Within the scope of this application, the term % v/v relates to a percentage by volume. Unless otherwise specified, where reference is made to a solution, water is used herein as solvent for the solutions. A 100 mL solution with 5% v/v glutaraldehyde contains, accordingly, 5 mL of pure glutaraldehyde (preferably with an absorption ratio of 235 nm:280 nm<0.5.) The term % w/v relates within the scope of this application to a proportion by weight. 100 mL solution with 0.9% w/v sodium chloride contains, accordingly, 0.9 g of sodium chloride.

In accordance with one embodiment of the method according to the invention the cross-linking agent is a glutaraldehyde-containing solution preferably including 0.01% v/v to 2% v/v glutaraldehyde, preferably in DPBS without Ca/Mg. Alternatively, other aqueous buffers without Ca/Mg known to a person of ordinary skill in the art to which the invention belongs can be used. Furthermore, other solutions can be used which contain a cross-linking agent selected from the group of glutaraldehyde, carbodiimide, formaldehyde, glutaraldehyde acetals, acyl azides, cyanimide, genepin, tannin, pentagalloyl glucose, phytate, proanthocyanidin, reuterin and epoxy compounds.

In accordance with one embodiment of the method according to the invention, the tissue arranged or clamped in the mold is exposed to the cross-linking agent for 1 to 3 days, preferably 2 days, at 2° C. to 10° C., preferably at 4° C.

In accordance with one embodiment of the method according to the invention, the tissue is then exposed to the cross-linking agent for 10 to 18 days, preferably 14 days, preferably at room temperature (typically 20° C. to 25° C.), wherein the cross-linking agent is preferably changed every 1 to 3 days, preferably every 2 days.

In accordance with a further aspect of the invention, an implant is disclosed which includes a tissue which has been impressed with a 3D shape by means of the method according to the invention.

In accordance with a preferred embodiment of the implant, the implant is a heart valve prosthesis which includes an artificial heart valve formed from the tissue, which is fastened, preferably sewn, to an expandable or self-expanding main body implantable by catheter. An application for venous valves, prostheses, or vascular implants is also included.

Controlling the thickness of biological tissues, such as pericardial tissues, during the cross-linking with glutaraldehyde, as well as the three-dimensional shape thereof, facilitates the object of thinner tissues for TAVI systems having defined three-dimensional shapes. Not least as a result catheters having smaller overall diameters can be produced, which in turn contribute to fewer vascular problems during the insertion and positioning of the TAVI heart valves.

A further great advantage of the solution according to the invention is the trouble-free integration in existing production processes of thickness control by mechanical space limitation. The proposed possibility for arbitrary shaping of three-dimensional porous supports or mold regions initially works without the use of new chemical substances. It is achievable, as a result, that the mechanical properties and the chemical ingredients, which in the case of new substances potentially should be tested with regard to their compatibility, remain constant.

A further great advantage is the possibility of being able to define a three-dimensional structure for the tissue by means of the porous supports during the cross-linking which supports the subsequent function of the tissue as an implant. In particular with use of open-pore polymer supports or mold regions made of polymer, for example made of polycarbonate, complex geometries can be easily realized by rapid prototyping. This allows, for the first time, a shaping of the tissue adapted to the valve geometry, whereby completely new valve geometries are conceivable. Due to the three-dimensional shaping of the tissue, it is additionally possible to optimize the opening and closing behavior of the valve cusps.

A further great advantage is the possibility of three-dimensional shaping of the biological valve part already during the tissue processing and not at a later step by cutting and sewing. Inner seams are therefore eliminated which are necessary only for shaping and for connecting a large number of individual tissue pieces. Seams for fixing to the valve stent are still necessary, but the number thereof can be reduced by the inner stability of the tissue piece. Due to the new pre-shaping, the spectrum of valve designs that can be geometrically realized increases. It is thus also possible to positively influence the hemodynamics by means of an advantageous shaping. This works well in particular by the proposed method, since the number of seams and therefore also the number of knots, which influence the hemodynamics, is significantly reduced. Furthermore, the shape of natural heart valves can be recreated particularly well by the shaping proposed herein, which additionally contributes to the generation of natural hemodynamics. Trouble-free blood flow can thus be achieved, and wearout at the seams can be reduced. Optimized hemodynamics provided, the valve material as well as the surrounding tissue is also prevented from damages. Due to the reduction of the number of necessary seams, the number of potential weak points with regard to the longevity of the valve implant is also reduced.

In particular, embodiments in which, by means of the present method, a heart valve is manufactured from three similar pieces are also provided. In an embodiment of this type the valve is formed merely by three vertical seams, wherein the three commissure lines are mechanically loaded to the same extent. A significant reduction of the seams is thus achieved, as well as a uniformly loaded valve. Due to the enormous saving of seams, a valve which is manufactured merely from one tissue piece is also preferred

The present proposal does not rule out the fact that further treatment steps not simultaneously affecting and in particular not impairing the properties of the tissue, such as tissue geometry, tissue mechanics and cross-linkability, could also be carried out on the tissue that is to be cross-linked or is cross-linked. By way of example, a decellularization as described in the prior art, before the cross-linking method proposed herein, is also encompassed by the invention.

### DESCRIPTION OF THE DRAWINGS

The invention will be explained in greater detail hereinafter on the basis of exemplary embodiments with reference to the figures, in which:

FIG. 1 is a schematic view of a mold for reducing the thickness of tissue plates without (cross-section at the top) and with (cross-section at the bottom) three-dimensional shaping;

FIG. 2 is a schematic illustration of the lower mold region or mold matrix for the production of the complete biological tissue in one piece for a TAVI valve.

FIG. 3 is a graph showing the thickness reduction by clamping porcine pericardial tissue during cross-linking with 0.6% v/v glutaraldehyde between porous supports, here formed from borosilicate glass, with the pore sizes: P3: 16-40 μm; P4: 10-16 μm; P5: 4-5.5 μm compared to freely cross-linked porcine pericardial tissue.

FIG. 4 is a graph showing shrinkage temperatures of porcine pericardial tissue treated during cross-linking with 0.6% v/v glutaraldehyde by clamping between porous mold regions or supports (pore sizes: Pore 3: 16-40 μm; Pore 4: 10-16 μm; Pore 5: 4-5.5 μm) compared to native and to freely cross-linked porcine pericardial tissue.

### DETAILED DESCRIPTION

The method presented herein describes three-dimensional shaping of biological tissues, in particular during a simultaneous thickness reduction or control of the tissue (for example pericardial tissue) during fixing with glutaraldehyde or other cross-linking agents by cross-linking between porous mold regions of a mold. Due to this space limitation, it is possible to control or to reduce the thickness of the tissue during the fixing and to influence the physical form of the tissue. During fixing with reactive agents, a significant volume increase up to a factor of two is usually observed due to the resultant structural changes within the tissue. Shaping elements of a mold 1, referred to here as mold regions 3, are used and are produced for example with the aid of 3D printing from porous plastic, such as polycarbonate.

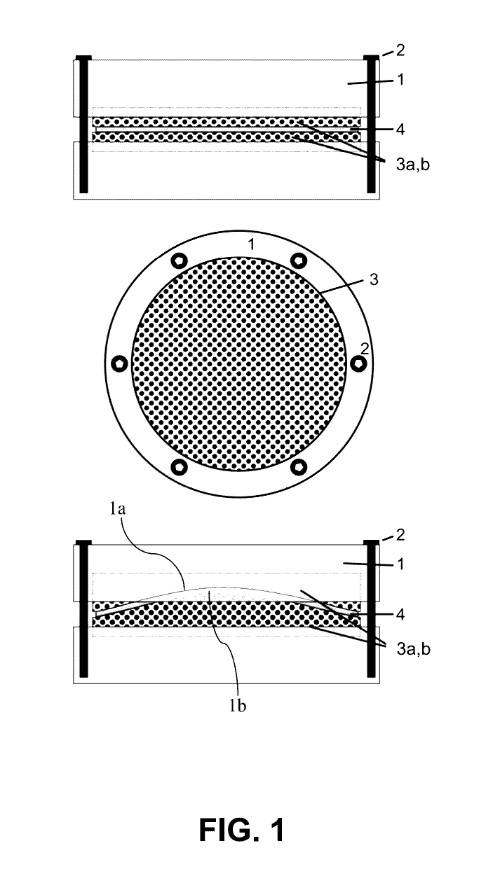

The used structure or the used mold 1 for thickness control and 3D shaping during the cross-linking is shown schematically in FIG. 1 (lower cross-section).

The mold then has a first 3D contact face 1a for laminar contact against an upper side of the tissue and also a second 3D contact face 1b for laminar contact against an rearside of the tissue 4, wherein the tissue 4 can be clamped between the two contact faces 1a, 1b so that it lies on both sides thereagainst and at the same time is to be cross-linked by means of a cross-linking agent so that the cross-linked tissue 4 has a 3D shape and, where applicable, a predefined thickness after removal from the mold.

The mold 1 here has an upper mold region 3a, which forms the first 3D contact face 1a, and a lower mold region 3b, which forms the second 3D contact face 1b. The mold regions 3 are permeable to the cross-linking agent so that this can reach the tissue via the mold regions during the cross-linking and can contact said tissue.

The tissue to be cross-linked is placed in the native state between the porous mold regions and is fixed in a manner bearing tightly thereagainst with the aid of a fixing 2 (for example clamps or screws).

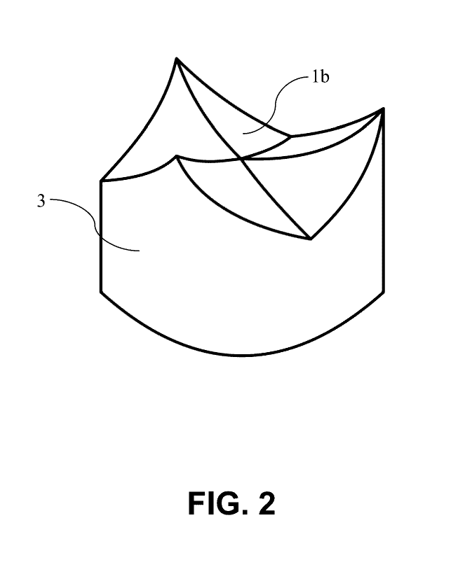

FIG. 2 schematically shows the lower mold region or part of the mold matrix for the biological tissue of a TAVI valve. By use of a matrix of this type, it is possible to produce the entire tissue part of a valve in one piece, which can be sewn into the valve stent. Shaping of one piece is achieved by the use of three (one for each cusp; not shown) mold pieces, which are placed from above onto the shown lower part 3 during the cross-linking. In this embodiment the entire valve consists for example of a single continuous tissue piece, which is provided with its end shape with just one vertical side seam. The upper edge of the tissue is led out, in the solution according to the invention, between the three upper mold parts and is cut off after the cross-linking (for example manually using surgical scissors or in a laser cutter for 3D objects). This avoids the formation of folds during the shaping. It is also possible to geometrically homogenize the tissue by defined tensioning. As already mentioned, a (one) pre-shaped tissue piece which still requires only a vertical side seam and sewing into the valve stent advantageously results from the method proposed herein. Production costs and time are significantly reduced by the proposed method and the resultant tissue pieces.

### EXAMPLE

An exemplary process for the thickness control and three-dimensional shaping of porcine pericardial tissue during the cross-linking with glutaraldehyde is described hereinafter.

Firstly, a pericardium from a pig is freshly removed in a slaughterhouse and is stored for 2 h at 4° C. in NaCl (0.9% w/v) with penicillin/streptomycin.

The pericardial tissue is then moistened in NaCl (0.9% w/v), wherein fat/connective tissue is removed and the tissue is cut to size.

The tissue is then rinsed in 100 ml NaCl (0.9% w/v) with slight movement.

The pericardial tissue is then clamped between the porous three-dimensional mold regions (polycarbonate), wherein the porous mold regions are saturated with NaCl (0.9% w/v).

The clamped tissue is then cross-linked in 100 ml glutaraldehyde solution (0.6% v/v in DPBS without Ca/Mg) for 48 hours at 4° C.

This is followed by a cross-linking in 100 mL glutaraldehyde solution (0.6% v/v in DPBS without Ca/Mg), more specifically for 14 days at room temperature (typically 20° C. to 25° C.), wherein the glutaraldehyde solution is replaced every two days with a fresh glutaraldehyde solution.

The pericardial tissue cross-linked in this way is removed from the porous three-dimensional mold regions or polycarbonate supports and is rinsed in 100 mL NaCl (0.9% w/v) at 37° C. with slight movement, more specifically 6× for 10 minutes.

The tissue can then be stored (preferably in 100 ml glutaraldehyde solution (0.6% v/v in DPBS without Ca/Mg)) or can be fed to further processing steps, such as a cutting to size.

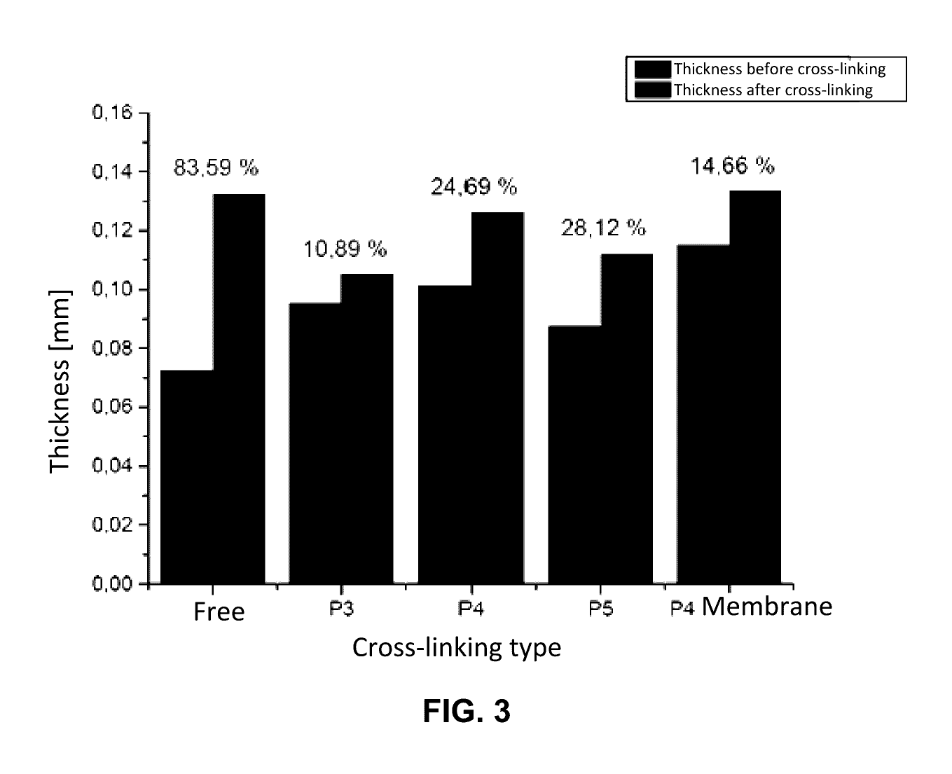

FIG. 3 shows the absolute thicknesses in mm and the thickness increase in % relative to the un-cross-linked starting state for various porous mold region materials (pore sizes: P3: 16-40 μm; P4: 10-16 μm; P5: 4-5.5 μm) and for freely cross-linked porcine pericardial tissue (n=10). The cross-linking without mold leads to a thickness increase of approximately 84%, which is a result of the incorporation of additional chemical bonds and the resultant structural changes of the collagen fibers. By clamping between the mold regions, this thickness increase is significantly reduced. This is achieved moreover without the degree of cross-linking of the tissue, measured via the shrinkage temperature, differing from a cross-linking without support materials.

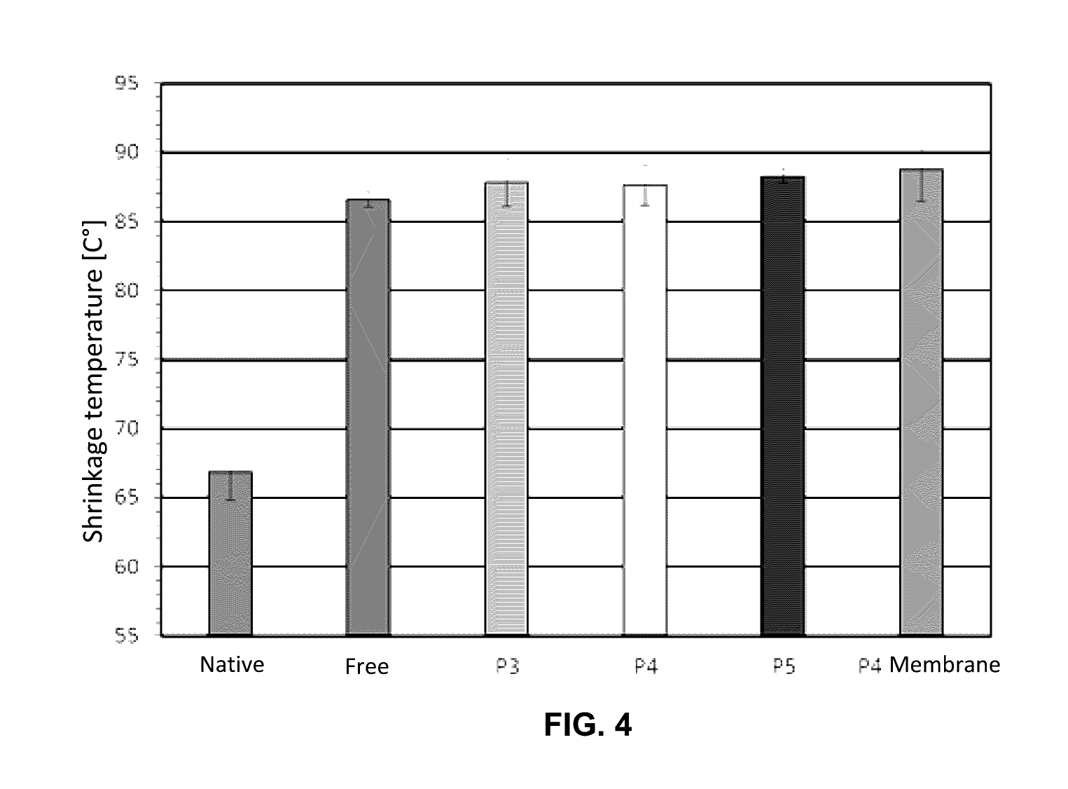

FIG. 4 shows the shrinkage temperatures of the tissue from FIG. 3, wherein ten samples in total were measured in each case and were removed from all regions of the cross-linked tissue area. The increase of the shrinkage temperature as a result of the cross-linking is clearly visible, which indicates the uniform cross-linking of all tissue, i.e. the glutaraldehyde is assured access to the tissue by the porous mold regions.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

What is claimed is:

1. A method for impressing a 3D shape onto a biological tissue (4), in particular pericardial tissue (4), the method comprising:
providing a mold (1) having a first 3D contact face (1a) for laminar contact against an upper side of biological tissue (4) and a second 3D contact face (1b) for laminar contact against a rear side of biological tissue (4);
arranging biological tissue (4) between and against the two contact faces (1a, 1b) while simultaneously cross-linking the biological tissue (4) by means of a cross-linking agent; and
removing the cross-linked tissue (4) from the mold.

2. The method of claim 1, characterized in that the thickness of the cross-linked tissue is limited to a maximum value by means of a spacing between the two 3D contact faces (1a, 1b).

3. The method of claim 1, characterized in that the mold (1) has an upper mold region (3), which forms the first 3D contact face (1a), and a lower mold region (3), which forms the second 3D contact face (1b).

4. The method of claim 3, characterized in that the mold regions (3) are permeable to the cross-linking agent, the method further comprising passing the cross-linking agent through the mold regions (3) to contact the tissue (4).

5. The method of claim 1, characterized in that the cross-linking agent is a glutaraldehyde-containing solution which optionally comprises 0.01% v/v to 2% v/v of glutaraldehyde, optionally in DPBS without Ca/Mg.

6. The method of claim 1, characterized in that the cross-linking agent contains a compound selected from the group consisting of glutaraldehyde, carbodiimide, formaldehyde, a glutaraldehyde acetal, an acyl azide, cyanimide, genepin, tannin, pentagalloyl glucose, phytate, proanthocyanidin, reuterin, and an epoxy compound.

7. The method of claim 1, characterized in that the step of crosslinking the biological tissue (4) comprises exposing the tissue (4) arranged in the mold (1) to the cross-linking agent for 1 to 3 days, optionally 2 days, at 2° C. to 10° C., optionally at 4° C.

8. The method of claim 7, further comprising exposing the tissue (4) to the cross-linking agent for an additional 10 to 18 days, optionally 14 days, optionally at room temperature, and optionally changing the cross-linking agent every 1 to 3 days, optionally every 2 days.

9. An implant comprising tissue (4) which has been impressed with a 3D shape by the method of claim 1.

10. The implant of claim 9, characterized in that the implant is a heart valve prosthesis which comprises an artificial heart valve formed from the tissue (4), which is secured, optionally sewn, to an expandable or self-expanding main body implantable by catheter.
